(19) United States
(12) Patent Application Publication
(10) Pub. No.: US 2008/0142024 A1
(43) Pub. Date: Jun. 19, 2008

(54) Athlete's foot cure aid

(76) Inventors: Jahangir S. Rastegar, Stony Brook, NY (US); Thomas Spinelli, East Northport, NY (US)

Correspondence Address: Thomas Spinelli, 2 Sipala Court, East Northport, NY, 11731, US

(21) Appl. No.: 11/998,927

(22) Filed: Dec. 3, 2007

Related U.S. Application Data
(60) Provisional application No. 60/872,191, filed on Dec. 2, 2006.

(51) Int. Cl. A61F 13/06; A61M 35/00; A45D 29/00

(52) U.S. Cl. 128/893; 604/293; 132/73

(57) ABSTRACT

A cure-aid for treating athlete's foot is provided. The cure-aid including: a bottom portion disposed on a bottom of at least one toe and one or more spacers connected to the bottom portion and disposed between at least two adjacent toes. The bottom portion and one or more spacers are formed of an absorbent material.

### CROSS-REFERENCE TO RELATED APPLICATION

This application claims the benefit of provisional application Ser. No. 60/872,191 filed on Dec. 2, 2006, the contents of which is incorporated herein by reference.

### BACKGROUND OF THE INVENTION

1. Field of the Invention

The present invention relates generally to devices and methods for reducing moisture in a region of the body and, more particularly, to devices and methods for curing athlete's foot.

2. Prior Art

Athlete's foot condition is caused by fungus growing between the toes and its immediate surrounding areas. The growth of the fungus is enhanced by the availability of moisture in its growth region and is inhibited by keeping the affected regions dry. In general, fungicides in the form of powder, liquid or gel are applied to the affected region for several days in order to eliminate the fungus infection. Various factors affect the speed with which the fungus infection is remedied. In some patients, particularly those who wear shoes and socks that minimize or even almost entirely prevent ventilation of the toes area, particularly if they have to keep shoes on continuously for hours at a time; or those with toes that fairly tightly press on each other, thereby minimizing ventilation of the region between the toes, particularly if the person lives in humid regions; once they are exposed to the fungus, it would quickly begin to grow and spread between the toes and cause a serious fungus infection. In such patients, the athlete's foot condition keeps on returning shortly after each treatment, particularly if they frequent places prone to fungus contamination, such as locker rooms and public shower areas in gyms.

Various powders, some medicated with anti-fungi medication are also available for use by those prone to fungi infection as a preventive measure or as medication at the outset of a fungus infection. However, such powders are effective for a relatively short period of time during which they absorb the present moisture and perspiration but saturate very quickly and become ineffective. In fact, due to the absence of ventilation and flow of air, they usually form a paste-like substance, stick to the regions between the toes, prevent whatever ventilation and airflow existed initially, and thereby provide a suitable region for fungus growth. In addition, the applied medicated or non-medicated powders are mostly forced out of the areas between the toes and the surrounding regions once the user begins to walk, mainly due to the generated motion and rubbing action between the toes and between the toes and surrounding regions and the environment (socks, shoes, and the floor if bare footed).

A need therefore exists for devices that would enhance ventilation between the toes, which could be used with anti-fungi medication and without medication, possibly with moisture controlling powders, to be used as preventive measures against fungi infection. Such devices would also assist in reducing foot order caused by perspiration and moisture and lack of ventilation and airflow.

### SUMMARY OF THE INVENTION

An objective of the present invention is to provide the means to allow ventilation between the toes and the surrounding regions. Hereinafter, “ventilation” also means the process of allowing flow of air without forcing the flow by a separate means. When the air is forced to flow between the toes and/or the surrounding regions, such as by a pumping action, the process will be referred to as “assisted ventilation.”

Another objective of the present invention is to provide one or more types of moisture absorbing medium, which may be solid or in the form of powder for the areas between the toes and surrounding areas, and providing the means to substantially keep the moisture-absorbing medium from sliding away from its intended positions as the result of walking and other activities.

Another objective of the present invention is to provide means to keep the medicated medium in contact with the affected area between the toes and its surrounding region and substantially prevent the medicated medium from sliding off as the result of walking and other activities.

Another objective of the present invention is to provide the means to provide assisted ventilation to the areas between the toes and its surrounding regions. The purpose of such assisted ventilation may be the prevention of fungus growth; accelerating the process of eliminating fungus infection by the medication; and/or the prevention of moisture and perspiration build-up and reduction of foot odor, in short a healthier foot and toes.

Accordingly, a cure-aid for treating athlete's foot is provided. The cure-aid comprising: a bottom portion disposed on a bottom of at least one toe; and one or more spacers connected to the bottom portion and disposed between at least two adjacent toes; wherein the bottom portion and one or more spacers are formed of an absorbent material.

The material can include one of pores and voids. The one of pores and voids can contain a powder-like medication for treating athlete's foot.

The cure-aid can further comprise an adhesive disposed on a bottom surface of the bottom portion to adhere to a sole of a shoe.

Each of the one or more spacers can include a spacer extension for wrapping around a top surface of a corresponding toe. The spacer extension can further comprise an adhesive on a surface thereof for adhering the spacer extension to the top surface of the toe.

The cure-aid can further comprise a pump for pumping air into at least a portion of the bottom portion and/or one or more spacers. The pump can be integral with the bottom portion. The pump can be disposed on a bottom surface of the bottom portion. The pump can include a cavity for holding air and one or more holes in communication with the cavity for discharging air from the cavity into at least the portion of the bottom portion and/or one or more spacers.

Also provided is a method for treating athlete's foot. The method comprising disposing a dry and absorbent material under and in between the toes of the foot while a user of the material is wearing shoes.

The material can include one of pores and voids and the method further comprises disposing a medicine for treating athlete's foot in the one of pores and voids.

The method can further comprise adhering the material to a sole of the shoes.

The method can further comprise wrapping the material around a top surface of a corresponding toe. The method can further comprise adhering the material to the top surface of the toe.

The method can further comprise pumping air into at least a portion of the material.

### BRIEF DESCRIPTION OF THE DRAWINGS

These and other features, aspects, and advantages of the apparatus of the present invention will become better understood with regard to the following description, appended claims, and accompanying drawings where:

FIG. 1 illustrates a side view of an embodiment of a cure-aid worn on a foot of a user.

FIG. 2 illustrates a front view of the cure-aid of FIG. 1 worn between the toe of the foot.

FIG. 3 illustrates another embodiment of a cure-aid.

FIG. 4 illustrates a variation of the embodiment of the cure-aid of FIG. 3.

FIG. 5 illustrates another embodiment of a cure-aid having a pump.

FIG. 6 illustrates the pump of FIG. 5.

### DETAILED DESCRIPTION OF THE PREFERRED EMBODIMENT

Although this invention is applicable to numerous and various types of skin conditions, it has been found particularly useful in the environment of treating athlete's foot fungus. Therefore, without limiting the applicability of the invention to treating athlete's foot fungus, the invention will be described in such environment.

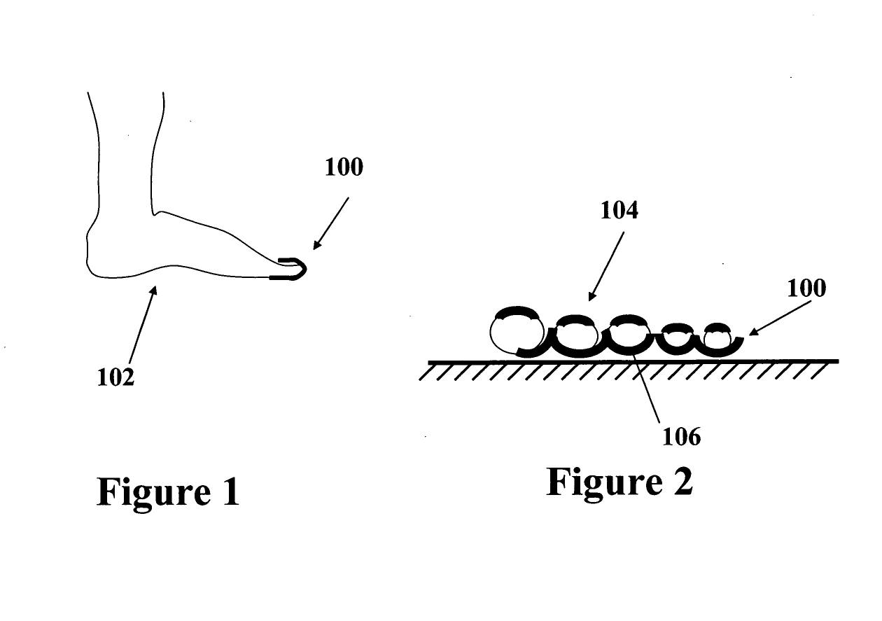

The general positioning of the present athlete foot cure-aid 100 over the toe areas of the foot 102 are shown in FIGS. 1 and 2. FIG. 2 shows the regions between the toes (spacer elements) and under the toes (bottom portions) that can be covered. The coverage may be extended to all the toes 104 or to those that are affected by the fungus infection or are to be protected from the fungus infection.

At least the portions of the various embodiments that are positioned between the toes and the surrounding area that have to be ventilated are made of materials preferably with one or more of the following characteristics:

1. Minimal material presence on the contact surfaces (minimal material area per surface area, such as a honeycomb) to allow maximum skin exposure to the airflow and medication (if any).

2. Maximum interconnected void to solid volume ratio to allow airflow.

3. The material can be highly absorbent.

4. The contact surfaces can be non-slippery over the skin surfaces, even with a certain degree of dampness.

5. For the medicated embodiments, the material can be able to hold powder-like medication in its pores and voids; or can be able to be treated with liquid or vapor or other type of medication, with the final material as close to being “dry” as possible.

The material can have enough strength to withstand several hours of wear and walking by the user. The product can be made to be disposable and worn for a full day.

The cure-aid 100 may be formed of any number of materials known in the art having one or more of the characteristics discussed above, such as paper or cotton based materials or similar synthetic materials or any combination thereof. The material can then be structured or is made with a structure that allows it to have one or more of the aforementioned characteristics, such as a honeycomb structure. Reinforcing fibers or the like may be added to the basic material to provide the required strength. Hereinafter, the material of the cure-aid is referred to simply as the cure-aid material.

The user can cover areas between the toes 104 and under the toes 104 as shown in FIG. 2 by way of a strip 106 of cure-aid material. The strip 106 may come as a continuous roll or may come cut to a certain length that can cover the foot size of an average user (the extra length may be used over the toes or may be cut off). FIG. 2 illustrates the strip 106 of cure-aid material having continuous half-loops such that each individual half-loop rests under each toe 104 and at least partially in-between each of the toes 104. Such a shape is exemplary and may take any number of forms and may also wrap around the top of the toes.

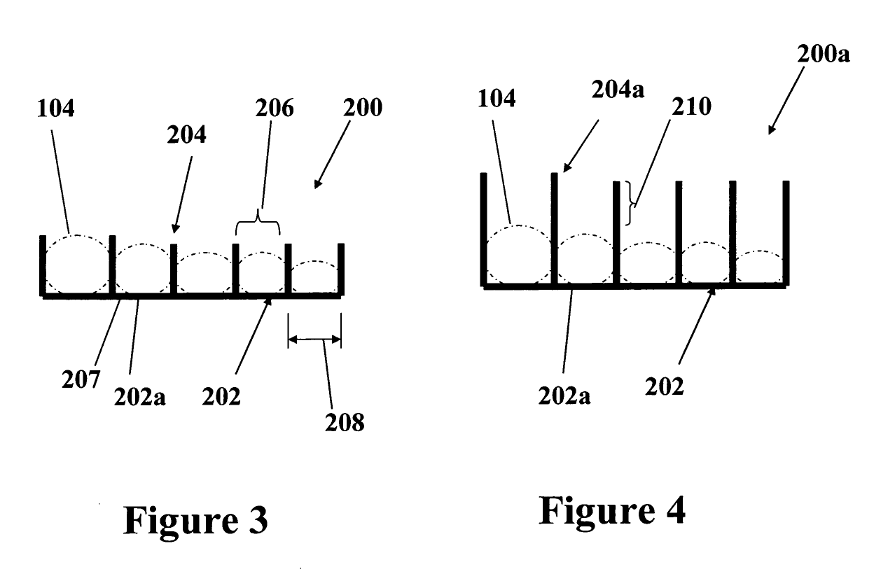

As shown in the front view of FIG. 3, the cure-aid 200 may come formed to fit the space between the toes 104 (shown in dashed lines) and cover the surface under the toes. In such a configuration, the cure-aid 200 can consist of a bottom portion 202, which covers the area under the toes 104, and spacer elements 204 that are positioned between the toes 104. The user can cut or break away some of the toe sub-elements 206 if less then all of the toes are to be treated for Athlete's foot or Athlete's foot prevented, such as at a weakened portion 207. The spacing 208 between the spacer elements 204 can be sized for an average person's toe sizes, for a man or woman's average toe sizes or may come in a variety of sizes.

The spacer elements 204a of the cure-aid 200a may be extended as shown in FIG. 4 to clear the toes 104 (shown in dashed lines). The extended spacer elements 204a may be made with the cure-aid material, in which case they can be bent to cover the top surfaces of the toes, or one or more spacer elements 204a may be partially or wholly made of adhesive tapes, such as a light adhesive, to affix the cure-aid 200a to the foot, such as to the top of the toes 104. The adhesive can be applied to only an end portion 210 of the spacer elements 204a where the remaining portions of the spacer elements 204a can be of the cure-aid material.

The configurations shown in FIGS. 2-4 may have an adhesive portion, which is positioned behind the bottom portion 202 and can be attached to the bottom of the foot behind the toe area to help keep the cure-aid in place. The adhesive portion can also be applied to the bottom 202a of the bottom portion 202 so as to affix the cure-aid 200, 200a to a sole of the shoe being worn.

The cure-aid material can also be formed in a glove like form, which is worn similar to socks. The front portion of such a design can be open to make it easier to be worn. The material may be provided with a certain amount of elasticity to allow it to fit a larger group of users and so that the cure-aid could be manufactured in a limited number of sizes.

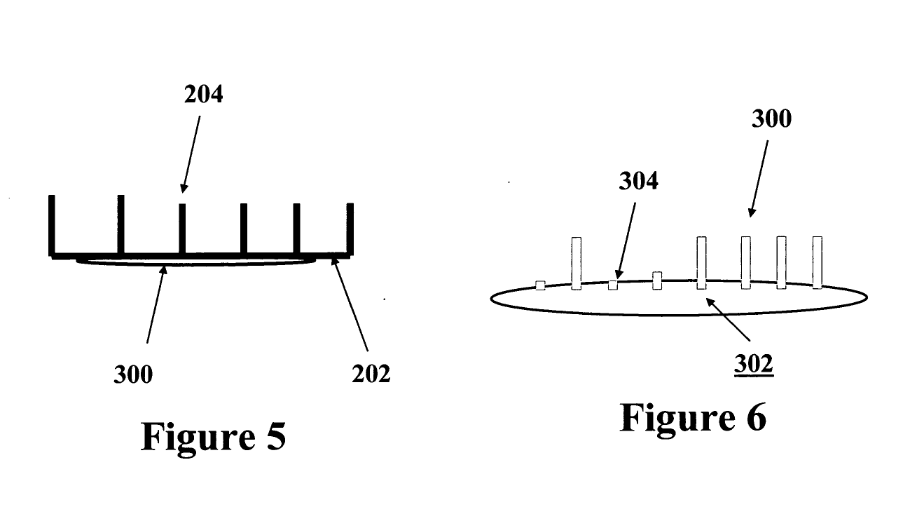

As shown in FIG. 5, any of the cure-aids discussed above can be provided with an air pump 300 (shown with the cure-aid 200 of FIG. 3 by way of example). The pump can pump air into areas between the toes and its surrounding regions as the user walks or runs. The air pump 300 can be very thin and positioned under the toes or immediately behind the toes as shown in FIG. 5. The pump 300 can be made out of synthetic materials such as a thin layer of plastic or elastomer (or combination thereof) with one or more openings discharging into the cure-aid material to pump a small amount of air into the bottom portion and/or the spacers of the cure-aid material.

The pump can be separately formed from the cure-aid or be an integral part of the cure-aid material. In the integral configuration, each time that the cure-aid material of the bottom portion and the spacers are compressed, they squeeze some of their entrapped air out of the material and suck in new air. In this configuration, the cure-aid material is preferably constructed such that air inflow from certain locations between the toes and between the toes and the bottom portion is provided with minimal resistance and outflow into the areas away from the aforementioned regions is also provided with minimal resistance. This is done to provide preferential flow out of the toe areas and inflow of more fresh air into these regions.

The pump 300, as shown in FIG. 6, includes a cavity to hold air and can be constructed from a resilient material, such as the plastic or elastomer so that it elastically deforms when pressure is applied due to a walking motion. During the elastic deformation of the pump, air in the cavity 302 of the pump is forced out through one or more openings 304 communicating with at least a portion of the cure-aid material.

While there has been shown and described what is considered to be preferred embodiments of the invention, it will, of course, be understood that various modifications and changes in form or detail could readily be made without departing from the spirit of the invention. It is therefore intended that the invention be not limited to the exact forms described and illustrated, but should be constructed to cover all modifications that may fall within the scope of the appended claims.

What is claimed is:

1. A cure-aid for treating athlete's foot, the cure-aid comprising:
a bottom portion disposed on a bottom of at least one toe; and
one or more spacers connected to the bottom portion and disposed between at least two adjacent toes;
wherein the bottom portion and one or more spacers are formed of an absorbent material.

2. The cure-aid of claim 1, wherein the material includes one of pores and voids.

3. The cure-aid of claim 2, wherein the one of pores and voids contains a powder-like medication for treating athlete's foot.

4. The cure-aid of claim 1, further comprising an adhesive disposed on a bottom surface of the bottom portion to adhere to a sole of a shoe.

5. The cure-aid of claim 1, wherein each of the one or more spacers includes a spacer extension for wrapping around a top surface of a corresponding toe.

6. The cure-aid of claim 5, wherein the spacer extension further comprises an adhesive on a surface thereof for adhering the spacer extension to the top surface of the toe.

7. The cure-aid of claim 1, further comprising a pump for pumping air into at least a portion of the bottom portion and/or one or more spacers.

8. The cure-aid of claim 7, wherein the pump is integral with the bottom portion.

9. The cure-aid of claim 7, wherein the pump is disposed on a bottom surface of the bottom portion.

10. The cure-aid of claim 10, wherein the pump includes a cavity for holding air and one or more holes in communication with the cavity for discharging air from the cavity into at least the portion of the bottom portion and/or one or more spacers.

11. A method for treating athlete's foot, the method comprising disposing a dry and absorbent material under and in between the toes of the foot while a user of the material is wearing shoes.

12. The method of claim 11, wherein the material includes one of pores and voids and the method further comprises disposing a medicine for treating athlete's foot in the one of pores and voids.

13. The method of claim 11, further comprising adhering the material to a sole of the shoes.

14. The method of claim 11, further comprising wrapping the material around a top surface of a corresponding toe.

15. The method of claim 14, further comprising adhering the material to the top surface of the toe.

16. The method of claim 11, further comprising pumping air into at least a portion of the material.
